(19) United States
(12) Patent Application Publication
(10) Pub. No.: US 2015/0328147 A1
(43) Pub. Date: Nov. 19, 2015

(54) METHOD FOR INTRODUCING BIOLOGICALLY ACTIVE SUBSTANCES INTO THE BRAIN

(71) Applicant: BBU-VITA CORP., Tortola (VG)

(72) Inventors: Dimitry TERTEROV, St. John's (CA); Sergei TERTEROV, Los Angeles, CA (US); Naum GOLDSTEIN, Berlin (DE); Roman GOLDSTEIN, Berlin (DE); Alexandr TERTEROV, Moscow (RU)

(21) Appl. No.: 14/419,724

(22) PCT Filed: Aug. 5, 2013
(86) PCT No.: PCT/DE2013/000458

§ 371 Date: May 15, 2015

(30) Foreign Application Priority Data
Aug. 5, 2012 (DE) 10 2012 015 248.5

(51) Int. Cl. A61K 9/00; A61K 31/197; A61K 31/13; A61K 31/485; A61K 47/02; A61K 38/22; A61K 38/50; A61K 38/21; A61K 39/395; A61K 31/137; A61K 38/28

(52) U.S. Cl. CPC A61K 9/0043; A61K 31/137; A61K 31/197; A61K 31/13; A61K 31/485; A61K 38/28; A61K 38/2264; A61K 38/50; A61K 38/21; A61K 39/3955; A61K 47/02

(57) ABSTRACT

The invention relates to a method for introducing biologically active substances into the brain, by the nasal introduction of a pharmaceutical composition consisting of biologically and therapeutically active substances together with membrane-active substances, hydrogen peroxide and nitrogen monoxide and their sources which remain in the nasal cavity in a disintegrated state, with only the pharmacologically active substances being transferred. The method is characterised in that the pharmaceutical composition is introduced nasally once or multiple times in complete or partial doses, that the time interval between introductions is between 3 and 180 seconds, preferably 60 seconds and that the drug dose is between 2 and 100 times smaller than the pharmaceutically predetermined dose.

The invention relates to the development of a medical-biological method for directly delivering synthetic and natural biologically active and medicinal substances from the nasal cavity into the brain on the basis of the interaction of these substances and membrane-active products of a free radical nature, and/or the sources of these products, with nerve and vessel structures of the nasal cavity.

The environment constitutes a source of molecules having multi-faceted biological activity which enter the organism along with food or from the surrounding air. The other important sources of these biologically active molecules are the internal sources of the organism, such as blood and interstitial fluid. To reach the organ structures or tissues, the biologically active molecules must pass through the biological membranes from the external and internal environments—for example the membrane structures of the brain.

Many biologically active molecules of the external and internal environment of the organism are useful and necessary for the normal functioning of the organs and tissue, including the brain. The biologically active molecules particularly include the majority of medications, as well as numerous biochemical compounds—the products of the metabolism, which form during metabolic processes in the organism.

At the same time, the delivery into the brain of many generally known and novel medications, the delivery of biotechnology products, and therapeutic uses of a range of metabolites in the treatment of the central nervous system (CNS), particularly the brain, constitute a serious problem. Methods for the delivery of several medications active in the CNS, from the nasal cavity into the brain, have been described in the scientific and patent literature of the past decade (Ming Ming Wen (2011) http ://www.discoverymedicine.com/Ming-ming-Wen/2011/06/13/olfactory-targeting-through-intranasal-delivery-of-biopharmaceutical-druqs-to-the-brain-current-development/; Costantino H. R. et al. (2007). Intranasal delivery—Physicochemical and therapeutic aspects. International Journal of Pharmaceutics 337: 1-24).

The works of Ming Ming Wen and Costantino et al. describe the known measures for the delivery of medicinal substances into the brain from the nasal cavity. These include the use of mucoadhesive excipients which facilitate passage through the membrane, such as liposomes, nanoparticles, and even the use of vasoconstrictors—for which there is no adequate scientific basis.

Several methods as described allow the passage of some small molecules into the brain, but still do not constitute a universal method for a wide spectrum of the medicinal substances.

The method of transnasal transport of medicinal substances into the peripheral blood and into the brain for the purpose of treatment and immunization (according to the patent EP 1 031 347 B 1) constitutes an obvious and known solution. The authors determine that the methods used to date for transnasal transport are not capable of ensuring a convincing principle for the comfortable and pleasant transport of pharmaceutically active substances through the membrane of the nasal cavity.

The authors believe that the presence of cytokines or their sources in the pharmaceutical formulas is an important element of the solution to the problem. The method used by the authors, however, only constitutes a possible pathway for a nasal delivery of the medication into the peripheral blood circulation and/or into the brain, and a means for immunization, and can only serve as a pharmacological and technological platform for a further improvement in the nasal delivery of the medication for the treatment of brain diseases.

The essential deficiencies of the described method are the complexity of the preparation and of the use of the medications, as well as a specific limitation for water-soluble compounds. A further deficiency of this method is the complexity of the preparation of the medicinal composition.

At an earlier date, we determined that free radical substances or their secondary products, hydrogen peroxide (H2O₂) or —NO active products, for example L-arginine, are capable of providing for an increase in the permeability of membrane structures of the nerves and blood vessels of the nasal cavity under certain conditions, and of contributing to the passage of biologically active substances into the brain (patent, DE 102 48 601; Eurasian patent no. 010692).

To date, formulations to solve the technical problem have been discussed in many regards, including the article Goldstein N., et al. 2012, Blood-Brain Barrier Unlocked. Biochemistry (Moscow), Vol. 77, No. 5, pp. 419-424. In this article, an experiment was carried out with tritium-marked dopamine (DA) to determine the efficacy of the introduction into the brain of the biologically active substance dopamine with the simultaneous nasal introduction of micromolar hydrogen peroxide. It is generally known that dopamine is not able to pass into the brain under normal conditions and trigger marked effects there.

To confirm the specific biological activity of dopamine in brain structures, animal experiments were conducted using the isotope product [³H]dopamine and using the neuroleptic haloperidol. It is generally known that dopamine is not able to pass into the brain under normal conditions and trigger marked physiological or therapeutic effects there. To confirm the specific physiological activity of dopamine in brain structures, animal experiments were conducted using the isotope [³H]dopamine and using the neuroleptic haloperidol.

The production of the tritium-marked [³H]dopamine used a reaction of the high-temperature solid-phase catalytic isotope exchange of the hydrogen for tritium in the dopamine hydrochloride preparation. The resulting [³H]DA preparation was purified in a Kromasil C18 column (8×150 mm) in the concentration gradient of an aqueous solution of acetonitrile in the presence of 0.1% heptafluorobutyric acid. The quantitative analysis of the preparation was carried out using HPLC and the Sigma-Aldrich DA standard (“Sigma/Aldrich”). The specific radioactivity of the evenly marked DA was 20 Ci/mol. The stock solution of the preparation contained 10⁻² M of the [³H]dopamine, with a volume activity of 0.75 mCi/ml.

In the preparation for the nasal introduction of the dopamine solution, the mixture was made ex tempore from the [³H]DA solution (concentration: 4×10⁻²; volume activity: 0.75 mCi/ml) [and an] isotonic solution of the stabilized hydrogen peroxide (Sigma-Aldrich). The final concentration of the dopamine and H₂O₂ in the solution introduced nasally was accordingly 10⁻² M and 10⁻⁵ M. A mixture of [³H]DA and H₂O₂ solutions was introduced in the experimental animal group. The animals in the control group received a mixture of [³H]DA and 0.9% NaCl.

In the preparation of the biological material from the rats, which were decapitated three minutes after the nasal introduction of the solutions containing [³H]DA, the brain was removed, the hypothalamus and both parts of the striatum were excised, and placed on a chilled surface (+4° C.). The removed brain structures were weighed within 35-40 s, frozen in liquid nitrogen, and placed into Eppendorf test tubes. The frozen samples were lyophilized for 48 hours and then extracted with 200 μL of 0.1M HClO₄ solution. Next, the samples were centrifuged within 15 minutes at 10,000 g and the supernatant was used for the determination of the [³H]DA and [³H]DOPAC.

Because the final concentrations of DA and DOPAC in the samples were not adequate for a determination of the radioactive derivatives of these substances, using UV detection, in connection with the peaks of the blood plasma, 10 μg of the unmarked standards of dopamine and its metabolite, 3,4-dihydroxyphenylacetic acid (DOPAC), were added to each of the extracts of the tissue for this reason prior to the chromatographic separation. The chromatographic analysis of the extract in 0.1M HClO₄ was carried out in a Kromasil C18 column, 5 μm (4×150 mm) at 20° C. using a gradient elution with acetonitrile (4-24%) in 0.1% heptafluorobutyric acid. The simultaneous detection of the wavelengths 254 nm and 220 nm was performed using a Beckman spectrophotometer (model 165, Altex). The sample volume was 100 μL. The fractions of the brain extracts of each animal of the experimental and control groups to which were added [³H]DA and [³H]DOPAC, separately, were analyzed quantitatively using liquid scintillation counting.

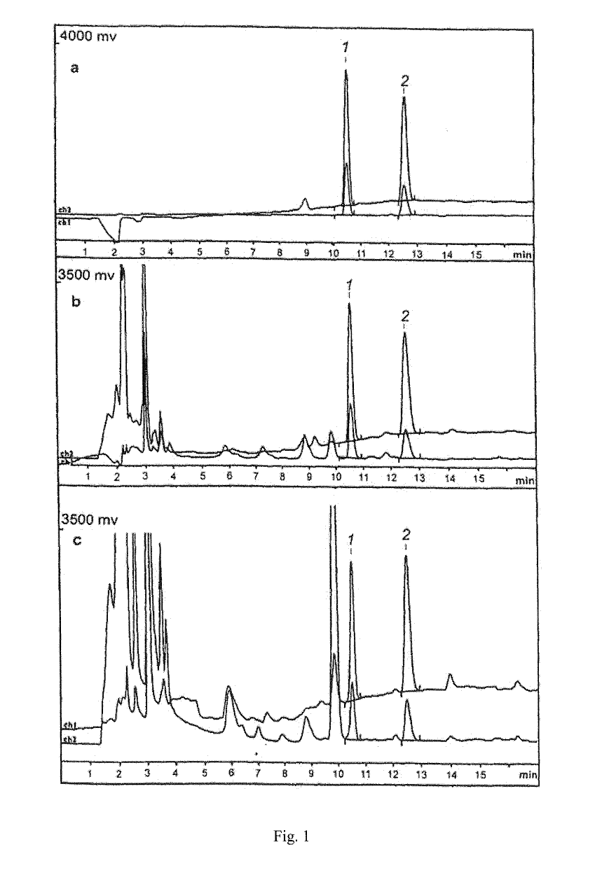

The typical chromatograms of the extracted solution of the hypothalamus and striatum extracts containing dopamine and DOPAC are illustrated in FIG. 1.

FIG. 1 The radioactivity of [³H]DA and [³H]DOPAC in chromatography extracted fractions of hypothalamus and striatum of rat brain.

Notes: The radioactivity of [³H]DA and [³H]DOPAC in the chromatography extracted fractions of hypothalamus and striatum, corresponding to the standards of the dopamine and DOPAC, were measured using a Tri-Carb 2900TR (Perkin Elmer) liquid scintillation counter. The radioactivity of the extracts of each of the eight rats of the control and experimental groups was measured as the number of radioactive decays per minute DPM); A conversion factor of 2.22×10⁶ was used for the conversion of these values into microcuries (μCi). The average efficiency of the counter was 49%. The conversion into an amount of dopamine was based on the concentration of the stock solution of 10⁻² M [³H]DA and the volume activity of 0.75 μCi/ml.

The cataleptic state in the rats was produced by a single intraperitoneal (i.p.) administration of haloperidol (“Ratiopharm”) at a dose of 0.25 mg/kg. The cataleptic response and the absence of reaction to external stimuli was confirmed as an 85% reduction of spontaneous motor activity. Next, the animals of the three separate rat groups were given a nasal administration of the solutions of 10⁻²M dopamine, 10⁻⁵M H₂O₂, or the DA+H₂O₂ mixture. The volume of the administered solutions was 50 μL in each nasal passage. The single dose given of dopamine was 0.8 mg/kg; the dose of H₂O₂ in this case was 34 ng/animal. To estimate the spontaneous activity of the rats, the “open field test” was used. The test site was a round arena with an 80 cm diameter, with a floor made of wood divided into 16 identical sectors and two concentric circles. The height of the barrier was 40 cm. To measure the spontaneous motor activity, the animal was placed in the center of the arena, and the number of horizontal movements between the sectors was recorded over 2 minutes. The observations began 90 s after the nasal administration of the preparations.

The research conducted on the lab animals was performed in compliance with the requirements of the ethics commission of the institute. 51 male “Wistar” rats were included in the experiment, with body masses ranging from 220-250 g. The animals were treated under standard vivarium conditions with unrestricted access to food and water. Over the course of three days prior to the start of the experiment, the rats were familiarized with contact with the researcher (“handling”).

The statistical significance (P) in the analysis of the number of radioisotope decays per minute was calculated using the non-parameterized one-sided Mann-Whitney test. The results were considered significant at P<0.05. In the investigation of the catalepsy model of the rats, the motor activity of the animals was expressed as conditional units of the visual registration. This led to an assumption of a non-Gaussian distribution of the results obtained.

The radioactivity of [³H]DA and [³H]DOPAC in the hypothalamus and striatum of the brain was significantly higher in the experimental groups of the rats which were administered the dopamine and H₂O₂ nasally and simultaneously in the form of a spray of the aqueous solution of the two components. The calculated concentrations of dopamine and DOPAC in the hypothalamus and striatum of the experimental and control rats are presented in Table 1. The control animals received the dopamine and a NaCl solution, rather than H₂O₂, in the spray (Tab. 1).

TABLE 1
The effect of micromolar H₂O₂ on the nasal delivery of dopamine into the structures
of the brain, in rats.

| Animal | Hypothalamus | | | | Striatum | | | |
|---|---|---|---|---|---|---|---|---|
| Group | [³H]DA^{a} | DA^{b} | [³H]DOPAC^{a} | DPOAC^{b} | [³H]DA^{a} | DA^{b} | [³H] DOPAC^{a} | DOPAC^{b} |
| I. | 14810 | 18.60 | 4653 | 6.31 | 24003 | 8.53 | 5327 | 1.73 |
| Experiment | [9687; 22558] | [16.66; 24.68] | [4112; 9523] | [5.64; 1076] | [20150; 26685] | [7.43; 9.99] | [4478; 6179] | [1.49; 2.11] |
| (n = 8); | | | | | | | | |
| [³H]DA + | | | | | | | | |
| H₂O₂ | | | | | | | | |
| I. Control | 285 | 0.41 | 111 | 0.15 | 887 | 0.31 | 214 | 0.07 |
| (n = 8); | [269; 302] | [0.37; 0.44] | [106; 123] | [0.13; 0.16] | [872; 1014] | [0.29; 0.33] | [197; 235] | [0.06; 0.08] |
| [³H]DA + | | | | | | | | |
| NaCl | | | | | | | | |
| Solution | | | | | | | | |
| I/II | 45.4 | | 42.1 | | 27.5 | | 24.7 | |
| P | 0.0004 | 0.0004 | 0.0004 | 0.0004 | 0.0004 | 0.0004 | 0.0004 | 0.0004 |

Notes:
[³H]DA: the tritium-marked dopamine;
[³H]DOPAC: the tritium-marked 3,4-dihydroxyphenylacetic acid;
^{a}the number of decays per minute;
^{b}3 pmol/mg of tissue.
The values are given as medians (1^{st} quartile; 3^{rd} quartile).The P values were calculated with the one-sided Mann Whitney Test. The volume of the solution introduced was 2 × 50 μL. The dose and number of the substances introduced: DA: 0.8 mg/kg; H₂O₂ - 34 ng.

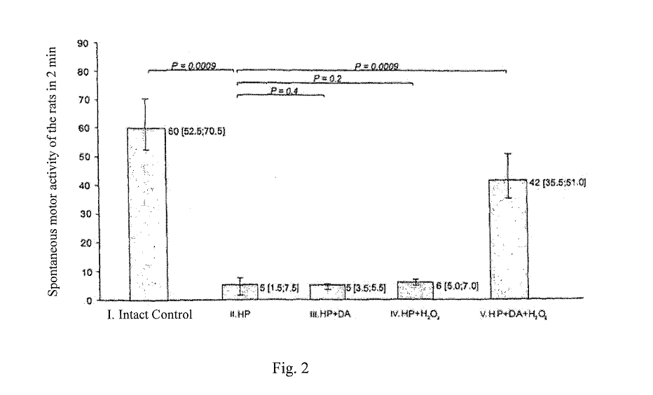

The injection of the haloperidol in the physiological experiments significantly suppressed spontaneous activity in the rats. The latency period for the onset of catalepsy following the i.p. administration of haloperidol was 9.4 [8.9; 9.8] minutes; the duration of catalepsy was 57.1 [54.8; 59.4]. The nasal introduction of the mixture DA+H₂O₂ resulted in a significant re-animation of spontaneous motor activity within 90 s. The separate administration in the control animals of the isotonic aqueous solution of dopamine or H₂O₂ did not re-animate the motor activity of the animals during the entire period of the catalepsy (FIG. 2).

FIG. 2: The effect of nasal administration of dopamine together with H₂O₂ on the spontaneous motor activity of the rats in the haloperidol catalepsy model.

Notes: HP: haloperidol; DA: dopamine: H₂O₂: hydrogen peroxide 10⁻⁵ M. A relative unit corresponds to crossing one sector in the “open field” test. The spontaneous motor activity of the rats: Group (I): intact control; (II): following i.p. application of HP; (III-IV) following nasal administration of the isotonic solution of DA or H₂O₂; (V): following the administration of the mixture of DA+H202. The values in the groups (III-V) were measured on the basis of the HP effect. The number of the rats in each group is n=7. The animals in each group (I to V) were tested separately. The values and the errors are presented as medians [1^{st} quartile; 3^{rd} quartile]. The P values were calculated using the one-sided Mann Whitney test.

For the example of dopamine as a test substance, the results showed that micromolar concentrations of H₂O₂ which have been administered nasally and at the same time as dopamine produce a rapid delivery of the dopamine into the structures of the brain. As such, only 3 minutes after the nasal administration, a significant increase in the dopamine content and the product of its metabolism, DOPAC, is observed in the hypothalamus and striatum. The dopamine peaks on the HPLC chromatograms of the extracts exactly matched the peaks of the dopamine standard. In the haloperidol catalepsy model, following the nasal administration of the dopamine in the mixture with H₂O₂, an increase was demonstrated for the dopamine content in the target organ, the striatum, such that the characteristic motor disturbances were effectively reduced in the experiment animals.

A nasal administration of [³H]DA together with the physiological solution in the control animals, in contrast, was neither accompanied by the increase in the dopamine content in the structures of the brain, nor by the reduction in the catalepsy effects.

The brief lifetime of the low molecular weight hydrogen peroxide H₂O₂ on the surface of the mucosa of the nasal cavity, as well as the maintenance of the effects over the relatively long period of time, suggest the involvement of a biochemical amplifier. We demonstrated at an earlier date (DE 102 48 601) that the candidate for this role can be the nitrogen monoxide radical (—NO), and that L-arginine (the substrate of the —NO-forming enzyme NO synthase (eNOS)) as well, administered nasally together with the dopamine, is able to reanimate spontaneous motor activity in the rates. At this point it is important to note that L-arginine is metabolized significantly more slowly in the nasal cavity than short-lived H₂O₂. The exact mechanism of the involvement of L-arginine in the delivery of the dopamine into the brain remains unclear.

The problem addressed by the invention is that of creating a method which eliminates the disadvantages of the prior art, wherein biologically and pharmaceutically-active substances in the form of medications are effectively and advantageously introduced directly into the brain following the nasal application by timed, quantitative, and intentionally sequential.

In the following experiments, which illustrate a continuation of the described investigations, the region of the effective concentrations of the hydrogen peroxide and L-arginine, as well as the time and quantitative parameters of the method, are presented.

The invention is explained and described using the following experiments:

Experiment 1. The nasal administration of the “dopamine+H₂O₂” mixture re-animates spontaneous motor activity in the rats following the intraperitoneal administration of haloperidol in a dose of 100 mg/kg body weight.

The substances (nasal): dopamine at a concentration of 10⁻³M with simultaneous concurrent nasal administration of hydrogen peroxide in various concentrations, in both nasal passages.

The criteria of the evaluation: the change in spontaneous motor activity as the sum of movements of the rats in different groupings of the animals, in the “open field” test.

The animal groups: control group: “intact control” (Group I), “haloperidol i.p.” (Group II), “haloperidol i.p.+dopamine” (Group III), and “haloperidol i.p.+H₂O₂” in various concentrations (Groups IV and V). Experimental groups: “haloperidol i.p.+dopamine+H₂O₂” in various concentrations (Groups VII-IX), (Tab. 2).

TABLE 2
spontaneous motor activity in the rats following intraperitoneal
administration of haloperidol and nasal administration of the
“dopamine + H₂O₂ mixture”.

| | | Spontaneous |
|---|---|---|
| No. | Animal Group | activity |
| I | Intact control (n = 7) | 35 ± 8 |
| II | haloperidol control i.p. (n = 9) | 3 ± 3 ^{##)} |
| III | haloperidol i.p. + nasal dopamine (n = 5) | 2 ± 1.7 ^{##)} |
| IV | haloperidol i.p. + nasal H₂O₂ (10⁻⁴M) (n = 5) | 1.8 ± 2.2 ^{##)} |
| V | haloperidol i.p. + nasal H₂O₂ (10⁻⁵M) (n = 6) | 3 ± 1.9 ^{##)} |
| VI | haloperidol i.p. + nasal dopamine + H₂O₂ (10⁻⁴M) | 35.4 ± 6.6 **) |
| | (n = 6) | |
| VII | haloperidol i.p. + nasal dopamine + H₂O₂ (10⁻⁶M) | 38 ± 7 **) |
| | (n = 7) | |
| VIII | haloperidol i.p. + nasal dopamine + H₂O₂ (10⁻⁸M) | 16 ± 6.5 *) |
| | (n = 6) | |
| IX | haloperidol i.p. + nasal dopamine + H₂O₂ (10⁻¹⁰M) | 4.1 ± 3.3 |
| | (n = 6) | |

Notes:
^{##)}: P _{(Groups II-V)} _{vs. Group I} < 0.01; *): P _{(Group VIII) vs. (Group I)} < 0.05; **) = P _{(Groups VI and VII) vs. (Groups II-V)} < 0.01; P _{(Group IX) vs. (Groups II-V)} < 0.1 = not significant.

These results demonstrated that the minimal effective concentration of the endonasal hydrogen peroxide H₂O₂ lies in the range from 10⁻⁸ to 10⁻¹⁰ M. The assumption was made that the maximum effective concentration for nasal hydrogen peroxide H₂O₂ is 5×10⁻⁴ M because damage to the nasal mucosa structures can occur in higher concentrations.

Experiment 2: The nasal administration of the “dopamine+L-arginine mixture re-animates spontaneous motor activity in the rats following the intraperitoneal administration of haloperidol in a dose of 100 mg/kg body weight.

The substances (nasal): dopamine at a concentration of 10⁻³ M with simultaneous and concurrent administration with L-arginine in various concentrations, in one nasal passage. The criteria of the evaluation: the change in spontaneous motor activity as the sum of movements of the rats in different groupings of the animals, in the “open field” test. The animal groups: control group: “intact control” (Group I), “haloperidol i.p.” (Group II), “haloperidol i.p.+nasal dopamine” (Group III), and “haloperidol i.p.+nasal L-arginine” in various concentrations (Groups IV and V). Experimental groups: “haloperidol i.p.+nasal dopamine+L-arginine” in various concentrations (Groups VI-VIII), (Tab. 3).

TABLE 3
spontaneous motor activity in the rats following intraperitoneal
administration of haloperidol and nasal administration of the
“dopamine + L-arginine mixture”.

| | | Spontaneous |
|---|---|---|
| No. | Animal Group | activity |
| I | Intact control (n = 8) | 37 ± 10 |
| II | haloperidol i.p. (n = 8) | 3.7 ± 3 ^{##)} |
| III | haloperidol i.p. + nasal dopamine (n = 6) | 2.6 ± 1.7 ^{##)} |
| IV | haloperidol i.p. + nasal L-arginine (10⁻³M) (n = 5) | 6.4 ± 3.4 ^{##)} |
| V | haloperidol i.p. + nasal L-arginine (10⁻⁵M) (n = 5) | 4.8 ± 2.7 ^{##)} |
| VI | haloperidol i.p. + nasal (dopamine + L-arginine | 2.9 ± 8 **) |
| | (10⁻¹M) (n = 5) | |
| VII | haloperidol i.p. + nasal (dopamine + L-arginine | 19 ± 6.1 **) |
| | (10⁻³M) (n = 6) | |
| VIII | haloperidol i.p. + nasal (dopamine + L-arginine | 6.2 ± 4.6 * |
| | (10⁻⁷M) (n = 6) | |

Notes:
^{##}: P_{(Groups II-V) vs. Group I} < 0.01; **: P _{(Groups VI and VII) vs. (Groups II-V)} < 0.01; P _{(Group VIII) vs. (Groups II-V)} < 0.1 = not significant.

These results indicated that the minimal effective concentration of the endonasal L-arginine lies in the range of 10⁻⁷ M.

The maximum effective concentration of L-arginine for nasal administration is 10⁻¹ M because higher concentrations may provoke the side effects of L-arginine.

A further important parameter in the physiological reaction of the receptors is time. The physiological reaction of the receptors of the nasal cavity depends highly on the duration of the stimulus. Over the course of a continuous stimulation, a reduction in the reaction is associated with physiological adaptation. This constitutes a significant problem because a receptor adaptation to the acting stimulus—for example caused by H₂O₂ or— —NO—sharply reduces the sensitivity of the receptors as well as dependent biological reactions (F. R. Schmidt, G. Thews, 1983. Human Physiology. Springer. Berlin—Heidelberg—New York).

This adaptation can reduce therapeutic efficacy of medicinal substances. No method is known to date for maintaining the sensitivity of nasal receptors during the activity of H₂O₂ and —NO. The method we have developed is based on a short-term, intermittent (interrupted) action on the mucosa of the nasal cavity by neuroactive substances—for example H₂O2 or —NO in a pharmaceutical composition with biologically and therapeutically active substances.

In our experiments, the use of this method significantly increases the effect of the substance phenobarbital following nasal administration synchronously with H₂O₂. The oral application of phenobarbital has been known for a long time for the treatment of epilepsy and/or sleep disorders (P. Kwan, M J. Brodie: Phenobarbital for the Treatment of Epilepsy in the 21st Century: A Critical Review. Epilepsia 2004;45:1141-1149). The disadvantages of this treatment are the undesired side effects, including nausea, dizziness, increase in P-450 activity in the liver, and interruptions in the metabolism of many medications.

Experiment 3: The effect of phenobarbital was investigated experimentally on sexually mature white rats, using a nasal administration. The sleep duration conditioned by phenobarbital following nasal administration of phenobarbital was compared against the presence and absence of vaso-active and neuroactive substance. The typical results of an experiment are listed in Tab. 4.

TABLE 4
Comparison of the duration of sleep in rats following a single
administration and fractionated nasal administration of phenobarbital in
conjunction with the neuroactive substance hydrogen peroxide (H₂O₂).

| | Duration of the experimental sleep | |
|---|---|---|
| | (Minutes following the last administration) | |
| | single nasal application | triple nasal application |
| | of the full dose | of partial doses |
| Animal Groups | 18 mg | 3 × 6 (mg) |
| Control group | 128 ± 11.1 | — |
| (n = 3) | | |
| Experimental group | — | 161 ± 21.1 |
| (n = 4) | | |

Notes:
The dosage of phenobarbital was 90 mg/kg body weight; the total volume of the solution applied nasally was 2 × 100 μL; the concentration of H₂O₂ was 10⁻⁵M; the interval between successive nasal administrations in the experimental animal group was 60 s.

It can be contemplated that the number of applied partial doses and the pauses between successively administered partial doses is specific to the substance and/or the application, wherein these can range from 1 to 5 partial doses and/or 10 to 60 s, respectively.

The invention has the following advantages:
a direct delivery of medicinal substances to the brain,
a possibility of creating a wide spectrum of medications to treat illnesses of the CNS,
a possibility of a comprehensive and effective use of generic substances (a “third life” for generics),
increased therapeutic efficacy of medications compared to the methods known to date,
a significant drop in the effective dose of the medications and of the risks of undesired side-effects,
a reduction in the environmental burden of biologically active substances and their decomposition products.

1. A method for introducing biologically active substances into the brain by nasal administration of a pharmaceutical composition comprising pharmaceutically active substances together with membrane-active substances, hydrogen peroxide or a source thereof, and nitrogen monoxide or a source thereof, which remain in the nasal cavity in decomposed form, wherein only the pharmaceutically active substances are conveyed, characterized in that the pharmaceutical composition is administered one or more times nasally in full and/or partial doses, and a time interval between the administrations is between 3 to 180 seconds and a drug dosage is 2 times to 100 times smaller than the pharmaceutically specified dosage.

2. A method according to claim 1, characterized in that the pharmaceutically active substances are administered synchronously and/or alternatingly in one nasal cavity and/or in both nasal cavities, and the number of the nasal administrations is 1 to 5.

3. A method according to claim 1, characterized in that the pharmaceutically active substances produce a regulatory and therapeutic effect on the functions of the central nervous system and are administered in the form of synthetic and natural products and/or a composition of these materials with the membrane-active substances, hydrogen peroxide or source thereof, and nitrogen monoxide or source thereof.

4. A method according to claim 1, characterized in that the pharmaceutically active substances are regulators of the neurotransmitter system of the brain.

5. A method according to claim 1, characterized in that the pharmaceutically active substances are modulators of the neurotransmitter system of the brain.

6. A method according to claim 1, characterized in that the pharmaceutically active substances are endogenous metabolites which have a regulatory effect on the functions of the central nervous system.

7. A method according to claim 1, characterized in that the pharmaceutically active substances act on the central nervous system and have a molecular mass less than 1 kDa.

8. A method according to claim 1, characterized in that the pharmaceutically active substances act on the central nervous system and have a molecular mass greater than 1 kDa.

9. A method according to claim 1, characterized in that the pharmaceutically active substances are introduced nasally in a composition of cells or cellular structures.

10. A method according to claim 1, characterized in that the pharmaceutically active substances are generic substances.

11. A method according to claim 1, characterized in that the pharmaceutically active substances are used nasally in a dose from 1% to 100% of the generally accepted dosage.

12. A method according to claim 1, characterized in that the membrane-active substance hydrogen peroxide is used nasally in a concentration of from 10⁻⁹ M to 10⁻³ M.

13. A method according to claim 1, characterized in that the membrane-active substance nitrogen monoxide NO· is used nasally in a concentration of from 10⁻⁷ M to 10⁻¹ M.

14. A method according to claim 1, characterized in that one or more of the pharmaceutically active substances is in the form of a gel, a salve, an oil, a suspension, a liposome, or a nanosome.

15. A method according to claim 1, characterized in that the pharmaceutical composition further comprises a pharmaceutically acceptable stabilizer, an antioxidants, a gel-forming material, a pH regulator, an osmotic regulator, an emulsifier, a solubilizer, or an antimicrobial agents.

16. A method according to claim 1, characterized in that the pharmaceutical composition is in the form of a nasal spray.

17. The method of claim 1, wherein the time interval between the administrations is about 60 seconds.

18. The method of claim 2, wherein the number of nasal administrations is about 3.

19. The method of claim 4, wherein the regulators of the neurotransmitter system of the brain are agonists and/or antagonists of receptors of dopamine, serotonin, histamine, or acetylcholine.

20. The method of claim 5, wherein the modulators of the neurotransmitter system of the brain are selected from the group consisting of γ-aminobutyric acid, akatinol memantine, and derivatives thereof.

21. The method of claim 6, wherein the endogenous metabolites which have a regulatory effect on the functions of the central nervous system are selected from the group consisting of inductors of the endogenous substances, hormones, amino acids, opioids, and proteins.

22. The method of claim 7, wherein the pharmaceutically active substances that act on the central nervous system and have a molecular mass less than 1 kDa are selected from the group consisting of dopamine, venlafaxine, amantadine, and trimeperidine.

23. The method of claim 8, wherein the pharmaceutically active substances that act on the central nervous system and have a molecular mass greater than 1 kDa are selected from the group consisting of insulin, galanin-like peptides, leptin, L-asparaginase, interferons, and bevacizumab.

24. The method of claim 9, wherein the composition of cells or cellular structures includes stem cells, immune complexes, or monoclonal antibodies.

25. The method of claim 12, wherein the membrane-active substance hydrogen peroxide is used nasally in a concentration of from about 5×10⁻⁴ M to about 10⁻⁶ M.

26. The method of claim 13, wherein the membrane-active substance nitrogen monoxide —NO is used nasally in a concentration from about 10⁻⁴ M to about 10⁻¹ M.
